(19) United States
(12) Patent Application Publication
(10) Pub. No.: US 2025/0332347 A1
(43) Pub. Date: Oct. 30, 2025

(54) PLUNGER FOR AUTO-INJECTOR AND RELATED METHODS OF USE

(71) Applicant: Regeneron Pharmaceuticals, Inc., Tarrytown, NY (US)

(72) Inventors: Bryan C. GRYGUS, Tarrytown, NY (US); Richard Jeff GILDERSLEEVE, JR., Maple Grove, MN (US)

(73) Assignee: Regeneron Pharmaceuticals, Inc., Tarrytown, NY (US)

(21) Appl. No.: 19/187,456

(22) Filed: Apr. 23, 2025

Related U.S. Application Data
(60) Provisional application No. 63/638,447, filed on Apr. 25, 2024.

(51) Int. Cl. A61M 5/20; A61M 5/315

(52) U.S. Cl. CPC A61M 5/20; A61M 5/3158; A61M 2005/206

(57) ABSTRACT

A plunger may include a body extending from a proximal end to a distal end, the body further including: a head at the proximal end, the head including a first end face and a first outer wall extending from the first end face; and a first recess extending into the body at the distal end, the recess being defined by an inner circumferential wall of the body, wherein the body further includes a plurality of protrusions extending radially inward from the inner circumferential wall.

### CROSS-REFERENCE TO RELATED APPLICATION(S)

This patent application claims priority to U.S. Provisional Patent Application No. 63/638,447 filed on Apr. 25, 2024, the entirety of which is incorporated herein by reference.

### TECHNICAL FIELD

This disclosure is directed to plungers for auto-injectors and related methods of use.

### Introduction

In various available auto-injectors, upon activation by a user, a needle is deployed, and fluid is delivered from the needle into the user. Fluid may be delivered to the needle as a result of an actuation of a plunger disposed in a cartridge containing the fluid.

### SUMMARY OF THE DISCLOSURE

A plunger may include a body extending from a proximal end to a distal end, the body further including: a head at the proximal end, the head including a first end face and a first outer wall extending from the first end face; and a first recess extending into the body at the distal end, the recess being defined by an inner circumferential wall of the body, wherein the body further includes a plurality of protrusions extending radially inward from the inner circumferential wall.

A medical device may include a plunger having a body extending from a proximal end to a distal end, the body further including: a head at the proximal end, the head including a first end face and a first outer wall extending from the first end face; and a first recess extending into the body at the distal end, the recess being defined by an inner circumferential wall of the body, wherein the body further includes a plurality of protrusions extending radially inward from the inner circumferential wall; and a screw configured to extend into the first recess and engage with the inner circumferential wall of the body, wherein the screw includes a plurality of extensions, wherein each extension is disposed between a pair of circumferentially-adjacent protrusions of the plunger.

### BRIEF DESCRIPTION OF THE FIGURES

The accompanying drawings, which are incorporated in and constitute a part of this specification, illustrate various examples and together with the description, serve to explain the principles of the disclosed.

Aspects of the disclosure may be implemented in connection with examples illustrated in the attached drawings. These drawings show different aspects of the present disclosure and, where appropriate, reference numerals illustrating like structures, components, materials and/or elements in different figures are labeled similarly. It is understood that various combinations of the structures, components, and/or elements, other than those specifically shown, are contemplated and are within the scope of the present disclosure.

Moreover, there are many examples described and illustrated herein. The present disclosure is neither limited to any single aspect nor example thereof, nor to any combinations and/or permutations of such aspects and/or examples. Moreover, each of the aspects of the present disclosure, and/or examples thereof, may be employed alone or in combination with one or more of the other aspects of the present disclosure and/or examples thereof. For the sake of brevity, certain permutations and combinations are not discussed and/or illustrated separately herein. Notably, an aspect or implementation described herein as “exemplary” is not to be construed as preferred or advantageous, for example, over other aspects or implementations; rather, it is intended to reflect or indicate the aspect(s) is/are “example” aspect(s).

FIG. 1 is a perspective exploded view of exemplary components of an auto-injector including a plunger, according to an aspect of the disclosure.

FIG. 2 is an elevation view of the plunger of FIG. 1, viewed from a plane corresponding to line 2-2 of FIG. 1.

FIG. 3 is a cross-sectional view of the plunger of FIG. 1, taken along a plane corresponding to line 4-4 of FIG. 2, with certain features redacted for clarity.

FIG. 4 is a cross-sectional view of the plunger of FIG. 1, taken along a plane corresponding to line 4-4 of FIG. 2.

FIG. 5 is a close up of FIG. 4 illustrating an enlarged view of a head of the plunger, according to an aspect of the disclosure.

FIG. 6A is a close up of FIG. 4 illustrating an enlarged side elevation view of a plunger protrusion, according to an aspect of the disclosure.

FIG. 6B is close up of FIG. 4 illustrating an enlarged front elevation view of a plunger protrusion, according to an aspect of the disclosure.

FIG. 7 is a cross-sectional view of the plunger of FIG. 1 including dimensional designations.

FIG. 8 illustrates a cross-sectional view of the plunger of FIG. 1 taken from a plane corresponding to line 8-8 in FIG. 2, with the addition of a first screw of FIG. 1 engaged to the plunger.

FIG. 9A is a side view of a first screw of a screw assembly according to the disclosure.

FIG. 9B is bottom perspective view of a first screw of a screw assembly according to the disclosure.

FIG. 9C is a top perspective view of a first screw of a screw assembly according to the disclosure.

FIG. 9D is a cross-sectional view along line A-A of the first screw of a screw assembly shown in FIG. 9A according to the disclosure.

FIG. 10 illustrates a cross-sectional view of a pair of stacked plungers, according to an aspect of the disclosure.

FIG. 11 is a cross-sectional view of an auto-injector including a plunger, according to an aspect of the disclosure.

The present disclosure is neither limited to any single aspect nor example thereof, nor to any combinations and/or permutations of such aspects and/or examples. Each of the aspects of the present disclosure, and/or examples thereof, may be employed alone or in combination with one or more of the other aspects of the present disclosure and/or examples thereof. For the sake of brevity, many of those combinations and permutations are not discussed separately herein.

Notably, for simplicity and clarity of illustration, certain aspects of the figures depict the general structure and/or manner of construction of the various examples. Descriptions and details of well-known features and techniques may be omitted to avoid unnecessarily obscuring other features. Elements in the figures are not necessarily drawn to scale; the dimensions of some features may be exaggerated relative to other elements to improve understanding of the examples. For example, one of ordinary skill in the art appreciates that the cross-sectional views are not drawn to scale and should not be viewed as representing proportional relationships between different components. The cross-sectional views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

For ease of description, portions of example plungers described herein, and/or respective components, are referred to as proximal and distal portions. It should be noted that as to any one example plunger described herein, the term “proximal” is intended to refer to portions closer to a needle incorporated in an example auto-injector, and the term “distal” is used herein to refer to portions further away from such a needle. Similarly, extends “distally” indicates that a component extends in a distal direction, and extends “proximally” indicates that a component extends in a proximal direction.

The present disclosure is directed to various examples of plungers for an injection device (e.g., auto-injector) for self-administration of drugs, or other therapeutic agents, by a user.

Additional details of auto-injectors in accordance with the present disclosure can be found in PCT/US2018/031077 to Arnott, et al., filed on May 4, 2018, and published as WO 2018/204779 A1, and in U.S. application Ser. No. 18/055,895 to Grygus, filed on Nov. 16, 2022, the entireties of which are incorporated by reference herein. Additional details of vial piercing systems in accordance with the present disclosure can be found in U.S. Pat. No. 10,182,969, filed on Mar. 10, 2016, the entirety of which is incorporated by reference herein.

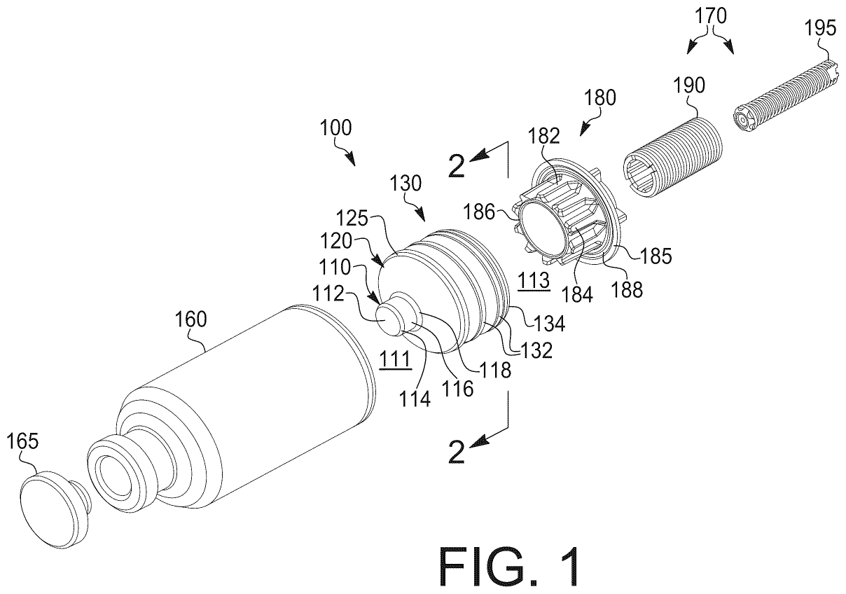

FIG. 1 is a perspective exploded view of exemplary components of an auto-injector including a plunger 100, according to an aspect of the disclosure. In one example, plunger 100 is defined by a body that includes or defines: a head 110 including a first outer wall 116; a second outer wall 120; and a third outer wall 130. Head 110 defines a proximal end 111 of plunger 100 and includes a first end face 112 bordered by a first outer edge 114 extending between first end face 112 and first outer wall 116. A base 118 of head 110 defines a transition between first and second outer walls 116, 120, as shown in FIG. 1. Second outer wall 120 diverges from base 118 towards third outer wall 130 and terminates at a second outer edge 125. In some examples, second outer edge 125 provides a transition between second outer wall 120 and a first rib 132 of a plurality of ribs 132 formed as part of third outer wall 130. In addition, an outer lip 134 may define an outer circumference of third outer wall 130, and thus plunger 100, at a distal end 113 of plunger 100.

In some examples, plunger 100 is configured to be positioned in a cartridge 160 through an end of cartridge 160 opposite an end that receives a stopper 165. In addition, plunger 100 may be configured to move within cartridge 160 with the actuation of a screw assembly 170 that may be coupled to (1) plunger 100, and (2) a translation mechanism (not shown). As shown, screw assembly 170 includes: a first screw 180; a second screw 190 configured to be coupled to first screw 180 with a first threaded coupling; and a third screw 195 configured to be coupled to second screw 190 with a second threaded coupling.

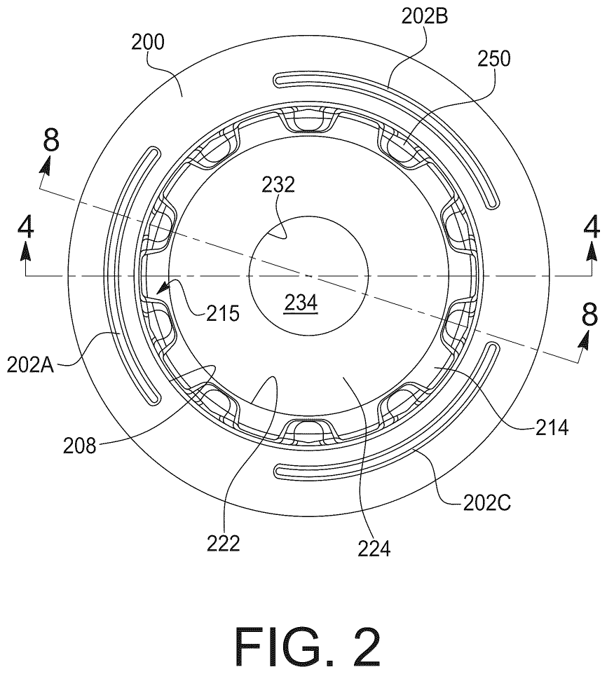

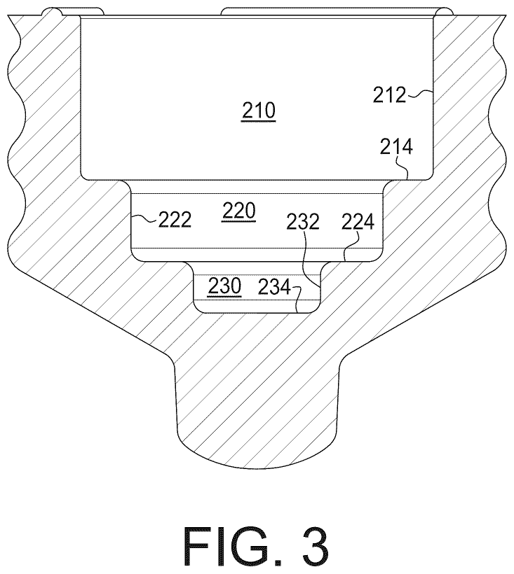

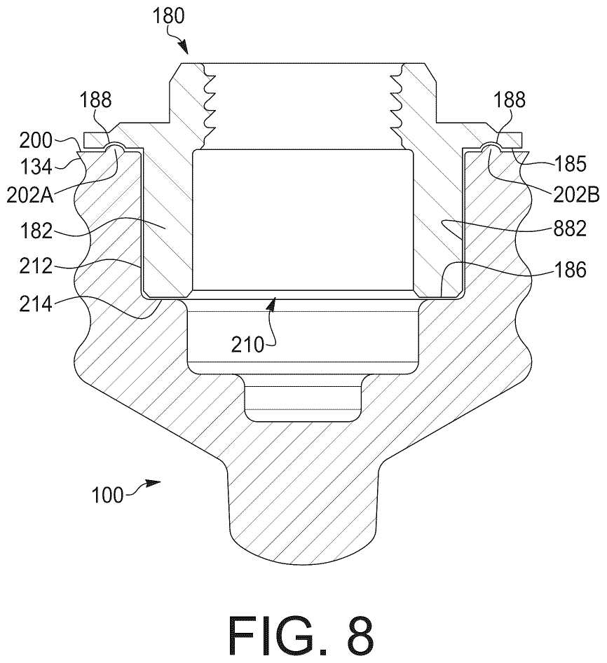

An outer portion of a body of first screw 180 defines a plurality of alternating screw protrusions 182 and screw slots 184. As described in more detail with reference to FIGS. 2-4, 6A, and 6B, plunger 100 is configured to engage or be engaged by screw protrusions and slots 182, 184 to affect a coupling between screw assembly 170 and plunger 100. In some examples, such as first screw 180 of FIG. 1, screw protrusions and slots 182, 184 may be surrounded by a flange 185 and extend from flange 185 to screw end face 186, or there between. In some examples, flange 185 may define a screw recess 188. In particular, screw recess 188 may be formed within flange 185 so as to also surround screw protrusions and slots 182, 184 as shown. In other examples, screw recess 188 may be configured into annular segments arranged in radial positions so as to correspond to end face protrusions (see. FIGS. 2-4 and 8) of plunger 100. More details regarding screw recess 188 in relation to example end face protrusions of plunger 100 are provided in the description below corresponding to FIG. 8.

FIG. 2 is an elevation view of plunger 100 of FIG. 1, viewed from a plane corresponding to line 2-2 of FIG. 1. More specifically, FIG. 2 depicts distal end 113 of plunger 100 which includes a second end face 200 and end face protrusions 202, 202A, 202B extending therefrom. The end face protrusions 202, 202A, 202B are substantially similar. As shown, one end face protrusion is designated with the numeral “202A” and another is designated with numeral “202B.” The designations 202A and 202B have been added for later reference in the description corresponding to FIG. 8, for the purposes of describing features related to these elements. References herein to “end face protrusion 202” may be construed as referencing one, any, each, and/or all of the end face protrusions of plunger 100.

As shown, second end face 200 surrounds a rim 208, which in turn surrounds a first recess 210 formed within the body of plunger 100. In particular, along a longitudinal axis of plunger 100 corresponding to a center thereof illustrated FIG. 2, first recess 210 is defined by surfaces of plunger protrusions 250 and inner circumferential wall 212 extending between plunger protrusions 250. Together, plunger protrusions 250 and inner circumferential wall 212 define a first recess wall from which a first end surface 214 extends inwardly to further define first recess 210.

First end surface 214 and second recess surface 222, provide a first step within plunger 100. Second recess surface 222 also defines a second recess 220 having a lesser diameter than first recess 210. When first recess 210 and second recess 220 have cross-sectional shapes that are non-circular, square or rectangular, for example, second recess 220 may have a smaller width that first recess 210. Second end surface 224 and a third recess surface 232, provide a second step within plunger 100. Third recess surface 232 also defines a third recess 230 having a lesser diameter (or width) than second recess 210. Third recess 230 is further defined by a third end surface 234 as shown in FIG. 2.

Referring back to first recess 210, in addition to collectively defining the first recess surface, plunger protrusions 250 and inner circumferential wall 212 define plunger slots 215 according to an aspect of the present disclosure. That is, spaces defined between circumferentially-adjacent pairs of plunger protrusions 250 and bordered by respective inner circumferential wall 212, define the above-mentioned plunger slots 215. Accordingly, plunger slots 215 may be configured to receive and be engaged by corresponding protrusions such as, screw protrusions 182, for example.

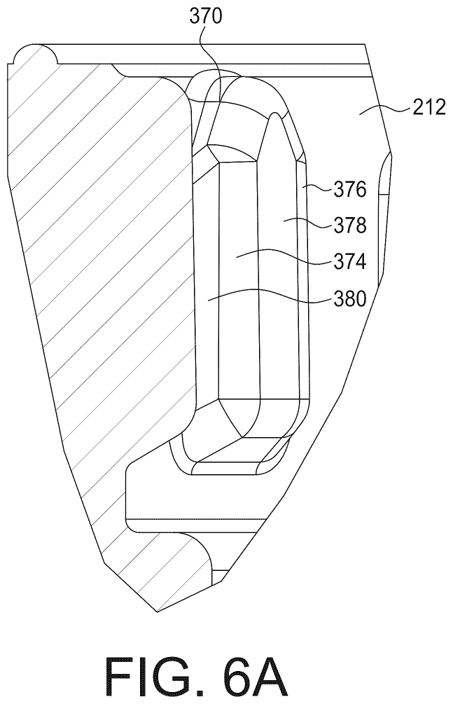

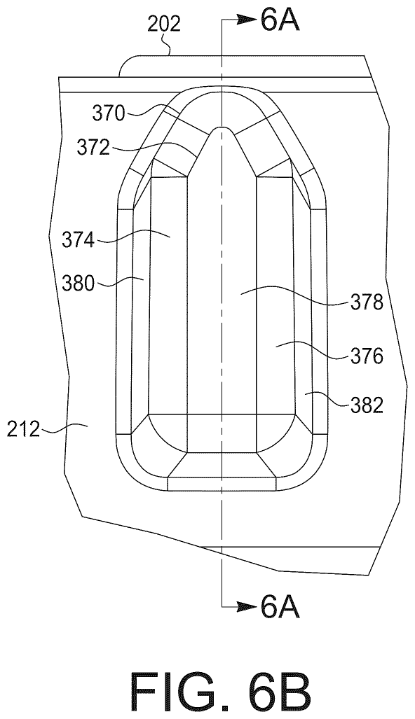

Referring to FIGS. 6A and 6B, each plunger protrusion 250 extends radially inward into first recess 210 from inner circumferential wall 212. Plunger protrusion 250 may include a blunted/pointed spear tip 370 facing the distal end of the plunger. Blunted spear tip may have a smaller width dimension than a remainder of plunger protrusion 250. Plunger protrusion 250 also may include a plurality of beveled surfaces, such as a curved or spear-shaped bevel 372. Bevels 374 and 376 extend from a radially-innermost surface 378 to bevels 380 and 382, respectively. Bevels 280 and 382 are closer to inner circumferential wall 212 than bevels 374 and 376, and in some embodiments are in direct contact with inner circumferential wall 212. Plunger protrusions 250, and particularly the blunted/pointed tip 370 may be self-centering relative to first screw 180. That is, the blunted/pointed tip 370 may act as an alignment/orientation feature, locating plunger protrusions 250 within the screw slots 184 of first screw 180.

It is contemplated that first screw 180 may engage with plunger 100 in other suitable manners. For example, first screw 180 may be coupled to plunger 180 by snap fit, press fit, adhesive or glue, notches, or the like. These disclosed mechanisms can be combined with one another and/or combined with the protrusions and extensions shown in the figures herein. For example, it is contemplated that in addition to the plunger protrusions 250 and screw protrusions 182, that adhesive and/or a snap fit connection may be added to supplement the connection between first screw 180 and plunger 100.

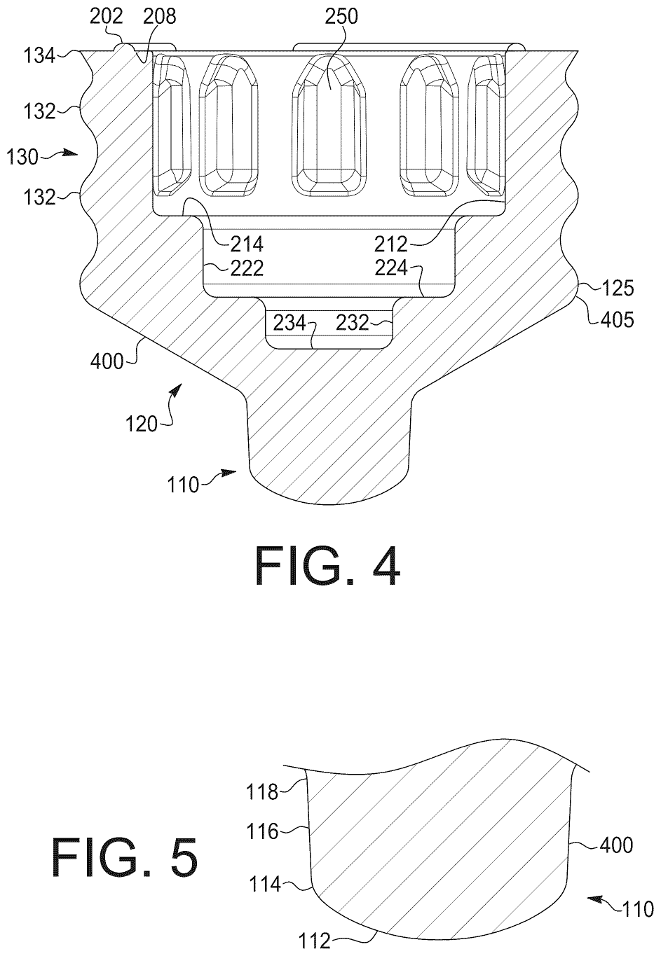

FIG. 4 is a cross-sectional view of plunger 100 of FIG. 1, taken along a plane corresponding to line 4-4 of FIG. 2. In some examples, a material layer 400 may be provided on head 110 and second outer wall 120. In addition, the material layer 400 may include a layer edge 405 disposed on second outer edge 125 of second outer wall 120. In still further examples, a portion of the layer edge 405 may also be disposed on a leading proximal portion of a first one of ribs 132 of third outer wall 130. According to some aspects of the present disclosure, material layer 400 may include a material such as a fluoropolymer material or other material that is highly resistance to solvents, acids, and/or bases. In other examples, material layer 400, and/or plunger 100 may include a fluoropolymer coated bromobutyl material. In still further examples, plunger 100 may include one or more rubber materials such as, e.g., halobutyls (e.g., bromobutyl, chlorobutyl, florobutyl) and/or nitriles, among other materials.

As illustrated in FIGS. 2 and 4, in some examples, plunger protrusions 250 may be evenly spaced about a circumference of first recess 210. In other examples, plunger 100 may include different numbers and spatial distributions of plunger protrusions 250. One of ordinary skill in the art will recognize that an arrangement of plunger protrusions 250 may be configured according to a configuration of screw protrusions for a screw, such as screw protrusions 182 of first screw 180, and vice versa. In other examples, a spatial arrangement, number, and structure of plunger protrusions 250 may be configured in agreement with other types of driven members having different engaging structural features. In the example shown, there are 10 protrusions, but more of fewer protrusions may be used, e.g., 2, 3, 4, 5, 6, 8, 12, 14, or more.

In practice, where plunger 100 is coupled to screw assembly 170, each of the first, second, and third recesses 210, 220, 230 may receive, or otherwise accommodate, a component of screw assembly 170. More specifically, in a fully retracted state of screw assembly 170, first screw 180 may extend longitudinally over a length of, and be surrounded by, the first recess surface defined by inner circumferential wall 212 and plunger protrusions 250. In addition, an end of first screw 180 may be position proximate to first end surface 214. Also, in the fully retracted state, a portion of second screw 190 may be disposed in second recess 220 surrounded by second recess surface 222, with an end of second screw 190 positioned proximate to second end surface 224. In addition, a portion of third screw 195 may be disposed in third recess 230 surrounded by third recess surface 232, with an end of third screw 195 positioned proximate to third end surface 234.

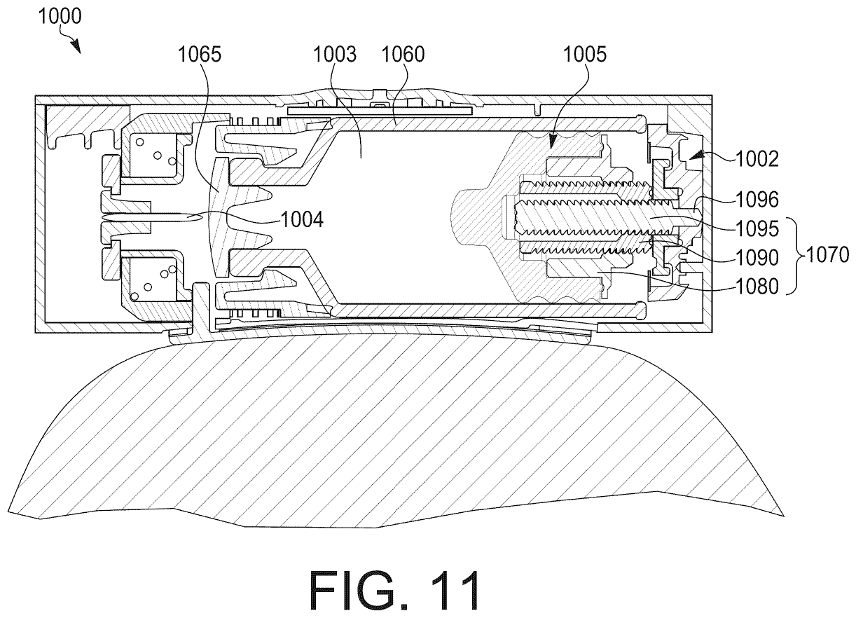

FIG. 5 is a close up of FIG. 4 illustrating an enlarged view of head 110 of plunger 100, according to an aspect of the disclosure. As shown, material layer 400 is disposed on head 110. First end face 112 may include a rounded protruding shape from which the first outer edge 114 extends-first outer edge 114 defining a curved edge that transitions to the first outer wall 116. In examples, the rounded protruding shape of the first end face 112 may reduce dead volume within example cartridges, such as cartridge 160 of FIG. 1 and cartridge 1060 (see FIG. 11). In other words, head 110 may be designed to minimize space remaining inside a stopper of cartridge 160 or a neck of cartridge 160, at the completion of an injection. It is also contemplated that plunger 100 could be used with line seals or other closure types.

In some examples, the first outer wall 116 may be formed with a profile that diverges as it extends from the first outer edge 114 to the base 118. As shown, the base 118 provides an outwardly extending transition from first outer wall 116. In other examples, first end face 112 and first outer wall 116 may be configured with a different profile. For example, the first outer wall 116 may extend at a substantially right angle relative to a plane traversing first outer edge 114. In still other examples, a configuration of the head 110 may correspond to an internal structural configuration of a cartridge, such as cartridge 160.

Example plunger protrusions, such as plunger protrusions 250, according to some aspects of the present disclosure, enable improved and rapid assembly, disassembly, and replacement of components of example auto-injectors. A threaded engagement is not used between example plungers, such as plunger 100, and example screw assemblies, such as screw assembly 170. Rather, example plungers protrusions described herein engage with slots (e.g., interlock) of screw or other types of drive components to provide a secure coupling for the purposes of translating example plungers according to the present disclosure within cartridges. At the same time, example plungers according to the present disclosure can be de-coupled from example screw assemblies or other types of drive components by simply pulling one of these components away from the other.

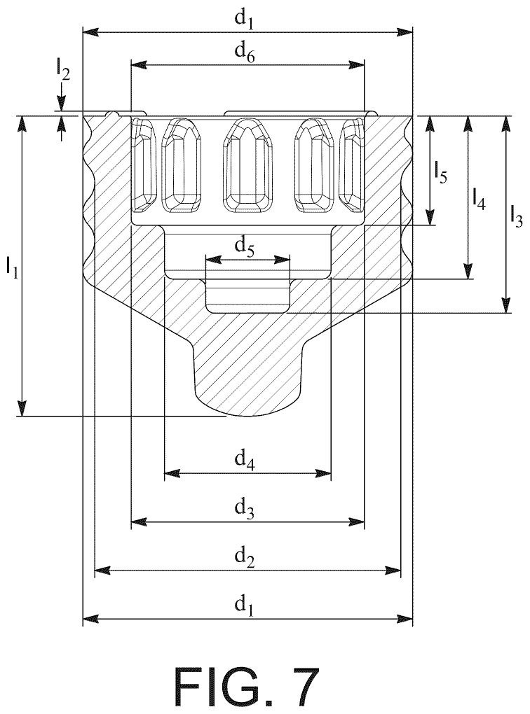

FIG. 7 illustrates various dimensions for exemplary plungers including dimensional designations for various structural aspects of plunger 100. Table 1 includes example measurements for the dimensions identified in FIG. 7. However, these dimensions are only intended to be exemplary.

TABLE 1
All Measurements in millimeters (mm)

| Designated | Example | |
|---|---|---|
| Dimension | Measurement | Variance (±) |
| l₁ | 17.69 | 0.1-1.2 |
| l₂ | 0.3 (max) | |
| l₃ | 11.8 | 0.05-0.6 |
| l₄ | 9.75 | 0.05-0.6 |
| l₅ | 6.5 | 0.05-0.6 |
| d₁ | 20.0 | 0.05-0.6 |
| d_{1′} | 20.35 (max) | |
| d₂ | 18.6 | 0.1-0.8 |
| d₃ | 14.0 | 0.05-0.6 |
| d₄ | 11.5 | 0.05-0.6 |
| d₅ | 5.0 | 0.05-0.6 |
| d₆ | 6.2 | 0.05-0.6 |

As shown in Table 1, each exemplary measurement may have an associated range of acceptable variances. A variance for l₁ may be in a range from 0.1 mm to 1.2 mm and may be, in particular, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, or any other value within the noted range. Variances for l₃, l₄, l₅, d₁, d₃, d₄, d₅, and d₆ may be in a range from 0.05 mm to 0.6 mm and may be, in particular, 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, or any other value within the noted range. While variances for l₃, l₄, l₅, d₁, d₃, d₄, d₅, and d₆ may fall within the same range, the respective variances need not be exactly the same for each dimension. Additionally, a variance for d₂ may be in a range from 0.1 mm to 0.8 mm and may be, in particular, 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.70 mm, 0.75 mm, 0.8 mm, or any other value within the noted range.

The disclosed plunger may be used for dispensing, for example, about 10 mL of fluid medicament from a cartridge, or may be used to dispense a different amount of fluid medicament, e.g., 1 mL, 2 mL, 3 mL, 4 mL, 5 mL, 8 mL, 12 mL or more. Furthermore, it is contemplated that the disclosed plungers could be scaled larger or smaller proportionally to be used in differently-sized cartridges.

While plunger 100 is shown herein used in conjunction with a telescoping motor/screw assembly, plunger 100 may be used in other configurations. For example, plunger 100 may be used with a syringe or injector having another suitable drive mechanism, such as, e.g., a plunger rod that is mechanically powered. Other drive mechanisms also may be used including, for example, a fluid (e.g., liquid or gas-powered device), or other mechanical means such as a spring or resilient member. It is further contemplated that plunger may be used in a device having a staked needle.

FIG. 8 illustrates a cross-sectional view of plunger 100 taken from a plane corresponding to line 8-8 in FIG. 2, with the addition of first screw 180 engaged to plunger 100. As illustrated, first and second end face protrusions 202A, 202B are received in screw recess 188. In some examples, end face protrusions 202 and screw recess 188 may be relatively sized such that end face protrusions 202 may snap fit into screw recess 188 when a sufficient force is applied to distal side of flange 185.

Incorporation of end face protrusions 202 is made easier with a width of second end face 200 resulting from the inclusion of outer lip 134. In addition, a surface of flange 185 and outer edge of first screw 180 may be disposed inside of a circumference of plunger 100 defined at distal end 113 by outer lip 134 and second end face 200.

FIG. 8 further illustrates an example arrangement between a portion of the first recess surface defined by one of the inner circumferential wall 212 and a screw protruding wall 882 of screw protrusions 182. As shown, in one example, inner circumferential wall 212 and screw protruding wall 882 may be configured (sized/shaped) such that screw protruding walls 882 engages (e.g., with sliding contact) corresponding inner circumferential wall 212 as a portion of the first screw 180 extending from flange 185 is positioned within first recess 210 of plunger 100. In addition, these structural features may be in abutment in a coupled state between plunger 100 and first screw 180, such as the coupled state illustrated in FIG. 8. The coupled state may also include screw end face 186 positioned proximate to first end surface 214. In other examples, screw end face 186 may be in abutment with first end surface 214 in a coupled state such as the coupled state illustrated in FIG. 8.

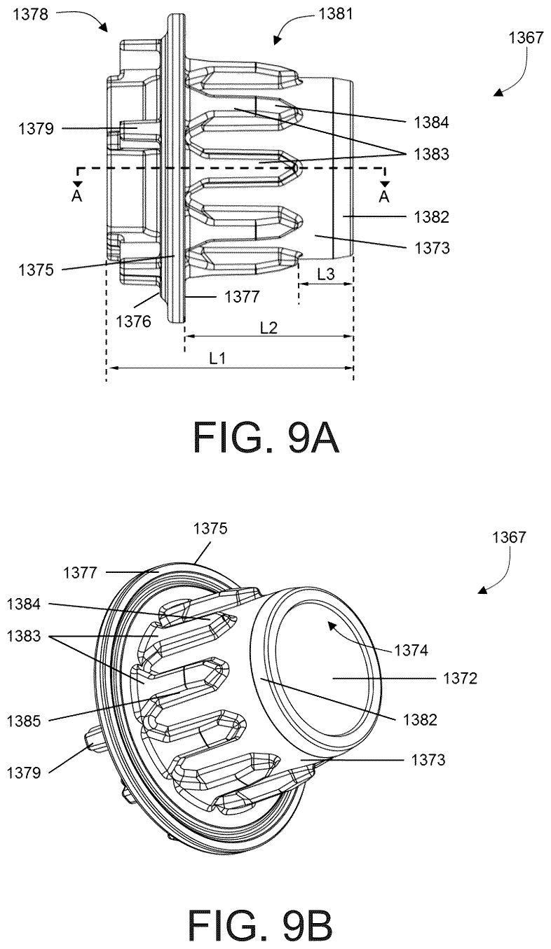

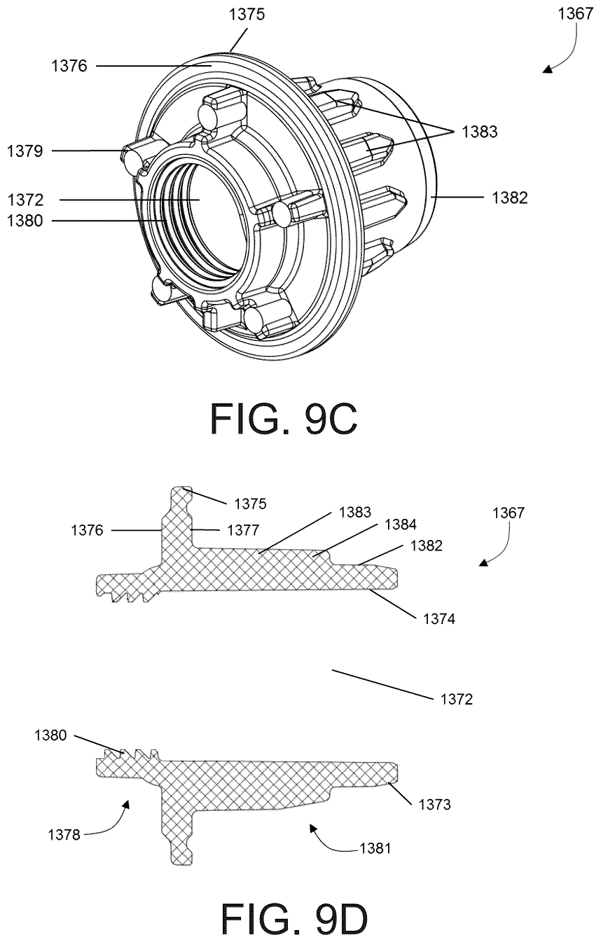

FIGS. 9A-9D depict an exemplary configuration of a first screw 1367 that may be used with plunger 100 in lieu of first screw 180. The first screw 1367 may generally comprise an upper portion 1378, a lower portion 1381, and a flange portion 1375. The flange portion 1375 may have a first surface 1376 and a second surface 1377 and separates the upper and lower portions 1378, 1381 of the first screw 1367. A conduit 1372 extends the length of the first screw 1367 and generally has a uniform diameter.

The upper portion 1378 may generally comprise one or more projections 1379 that protrude from the first surface 1376 of the flange portion 1375. In some embodiments, the one or more projections 1379 may be formed or partially formed by ejector pins during a molding process and are generally configured to fit into corresponding features of an output gear of a motor assembly. It should be understood that there may be any number of the one or more projections 1379 and that the one or more projections 1379 may be of any suitable shape and/or size. In some embodiments, there are at least two projections, four projections, or five projections.

The lower portion 1381 may generally comprise a nose region 1382 and one or more protrusions 1383 that extend radially outward from the exterior surface 1373 of the conduit 1372 and longitudinally from the second surface 1377 of the flange portion 1375. Each of the one or more protrusions 1383 generally comprises a tip 1384 and a surrounding side edge 1385. The one or more protrusions 1383 are configured to interlock with corresponding features found on the interior of plunger 100. For example, the one or more protrusions 1383 may be configured to nest within plunger slots 215 of plunger 100. The side edge 1385 features a chamfered design and the tip is generally tapered to facilitate nesting of the first screw 1367 into the plunger 100 during the assembly process. The nose region 1382 extends from the tips 1384 of the one or more protrusions 1383 to the end of the lower portion 1381. The nose region 1382 may include a chamfer toward the end of the lower portion 1381 to further facilitate nesting of the first screw 1367 within rim 208 of plunger 100. In some embodiments, nose region 1382 or a portion thereof may nest within second recess 220 of plunger 100 following assembly.

The configuration of the first screw 1367, including dimensions and tolerances thereof may be selected to balance the ease of assembly, the strength and durability of the coupling mechanisms, and the degree of deformation of the plunger 100 when under external pressure. For example, nose region 1382 may be configured to facilitate insertion of first screw 1367 into plunger 100. Moreover, tips 1384 may be shaped and sized so as to facilitate mating with plunger slots 215 and/or plunger protrusions 250. For example, tips 1384 may generally be triangular in shape with smooth edges, which may facilitate advancing of the protrusions 1383 past plunger protrusions 250 and into plunger slots 215. By facilitating insertion of first screw 1367 into plunger 100 during assembly, dislodging of plunger 100 from its position within cartridge 160 may be inhibited and an integrity of the seal of cartridge 160 may be maintained during assembly.

The length L1 of the first screw 1367 may range from about 12 mm to about 16 mm, including all sub-ranges and values there-between. In some embodiments, the length L1 of the first screw 1367 may range from about 12.5 mm to about 15.5 mm; from about 12.75 mm to about 15.25 mm; from about 13 mm to about 15 mm; or from about 14 mm to about 15 mm. In certain embodiments, the length L1 of the first screw 1367 may be about 14.42 mm, about 14.48 mm, about 14.54 mm, about 14.60 mm, about 14.66 mm, about 14.72 mm, about 14.78 mm, about 14.84 m, about 14.90 mm; or about 14.96 mm.

The length L2 of the lower portion 1381 of the first screw 1367 may range from about 8.0 mm to about 13.0 mm, including all sub-ranges and values there-between. In some embodiments, the length L2 of the lower portion 1381 of the first screw 1367 may range from about 8.0 mm to about 12.0 mm; from about 8.0 mm to about 11 mm; from about 8.0 mm to about 10.5 mm; from about 9.0 mm to about 12.0 mm; from about 9.0 mm to about 11.0 mm, from about 9.0 mm to about 10.5 mm; from about 9.5 mm to about 10.5 mm; or from about 9.9 mm to about 10.2 mm. In certain embodiments, the length L2 of the lower portion 1381 of the first screw 1367 may be about 9.62 mm, about 9.68 mm, about 9.74 mm, about 9.80 mm, about 9.86 mm, about 9.92 mm, about 9.98 mm, about 10.04 mm, about 10.10 mm, about 10.16 mm, about 10.22 mm, about 10.28 mm, about 10.34 mm, or about 10.40 mm.

The length L3 of the nose region 1382 of the lower portion 1381 of the first screw 1367 may range from about 0.5 mm to about 7.0 mm, including all sub-ranges and values there-between. In some embodiments, the length L3 of the nose region 1382 of the lower portion 1381 of the first screw 1367 may range from about 1.0 mm to about 4.5 mm; from about 1.5 mm to about 4.0 mm; from about 1.5 mm to about 3.5 mm; from about 2.0 mm to about 5.0 mm; from about 2.5 mm to about 5.0 mm; or from about 3.0 mm to about 5.0 mm. In certain embodiments, the length L3 of the nose region 1382 of the lower portion 1381 of the first screw 1367 may be about 5.41 mm, about 5.47 mm, about 5.53 mm, about 5.59 mm, about 5.65 mm, about 5.71 mm, about 5.77 mm, about 5.83 mm, about 5.89 mm, about 5.95 mm, about 6.01 mm, about 6.07 mm, about 6.13 mm, about 6.19 mm, about 6.25 mm, about 6.31 mm, or about 6.37 mm. The tolerances of the lengths described herein may be about 0.2 mm or less, about 0.15 mm or less, about 0.12 mm or less, about 0.10 or less, about 0.08 mm or less, or about 0.06 mm or less.

The threaded region 1380 of conduit 1372 may generally be characterized by several diameter measurements. For example, the threaded region 1380 may be defined by a minor diameter measured between two opposing crests of the threading, a major diameter measured between two opposing roots of the threading, or a pitch diameter measured between the midpoints of the threading on either side of the threaded region 1380. In some embodiments, the threaded region 1380 of the conduit 1372 may have a minor diameter ranging from about 6.75 mm to about 7.60 mm, a major diameter ranging from about 7.75 mm to about 8.40 mm, and/or a pitch diameter ranging from about 7.00 mm to 8.15 mm, including all sub-ranges and values there-between.

The minor diameter may range from about 6.75 mm to about 7.50 mm; from about 6.75 mm to about 7.40 mm, from about 6.75 mm to about 7.30 mm; from about 7.00 mm to about 7.50 mm; from about 7.00 mm to about 7.40 mm; from about 7.00 mm to about 7.30 mm; from about 7.15 mm to about 7.50 mm; from about 7.15 mm to about 7.40 mm; or from about 7.15 mm to about 7.30 mm. The major diameter may range from about 7.75 mm to about 8.30 mm; from about 7.75 mm to about 8.20 mm, from about 7.75 mm to about 7.10 mm; from about 7.90 mm to about 8.30 mm; from about 7.90 mm to about 8.20 mm; from about 7.90 mm to about 8.10 mm; from about 8.05 mm to about 8.30 mm; from about 8.05 mm to about 8.25 mm; from about 8.05 mm to about 8.20 mm; from about 8.05 mm to about 8.15 mm; or from about 8.08 mm to about 8.15 mm. The pitch diameter may range from about 7.00 mm to about 8.10 mm; from about 7.25 mm to about 8.00 mm, from about 7.25 mm to about 7.90 mm; from about 7.25 mm to about 7.75 mm; from about 7.25 mm to about 7.60 mm; from about 7.45 mm to about 8.10 mm; from about 7.45 mm to about 8.00 mm; from about 7.45 mm to about 7.90 mm; from about 7.45 mm to about 7.75 mm; from about 7.45 mm to about 7.60 mm; or from about 7.50 mm to about 7.60 mm. In certain embodiments, the minor diameter may be about 6.93 mm, about 7.00 mm, about 7.07 mm, about 7.14 mm, about 7.21 mm, about 7.28 mm, about 7.35 mm, about 7.42 mm, or about 7.49 mm; the major diameter may be about 7.78 mm, about 7.86 mm, about 7.94 mm, about 8.02 mm, about 8.10 mm, about 8.18 mm, about 8.26 mm, or about 8.34 mm, and the pitch diameter may be about 7.20 mm, about 7.26 mm, about 7.32 mm, about 7.38 mm, about 7.44 mm, about 7.50 mm, about 7.56 mm, about 7.62 mm, about 7.68 mm, about 7.74 mm, about 7.80 mm, or about 7.86 mm.

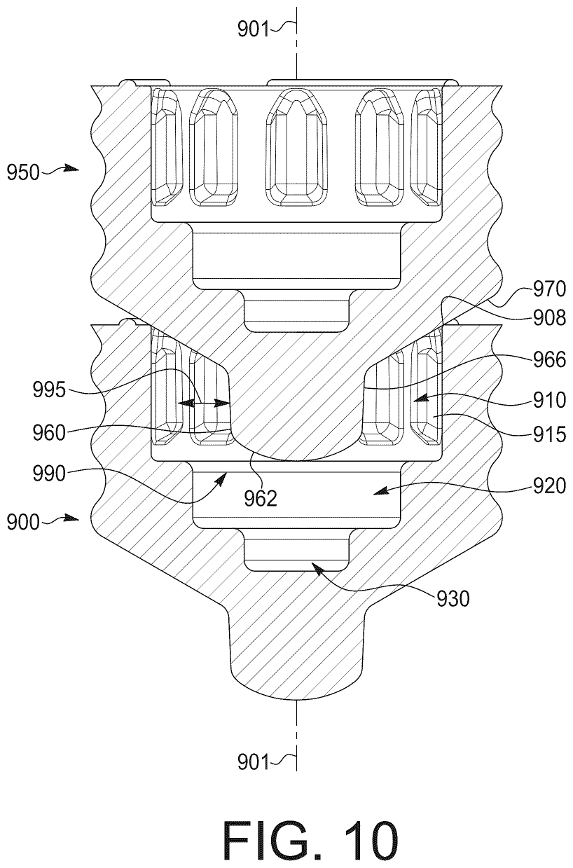

FIG. 10 illustrates a cross-sectional view of a pair of stacked plungers, according to an aspect of the disclosure. More specifically, a first plunger 900 is provided and a second plunger 950 is stacked on top of first plunger 900. A rim 908 of first plunger 900 is engaged with (e.g., supports), a second outer wall 970 of second plunger 950. As shown, with second outer wall 970 engage with or resting on rim 908, a head 960 of second plunger 950 extends into a first recess 910 of first plunger 900, but may not extend into a second recess 920 of the first plunger 900.

In some examples, rim 908 may define a chamfered surface having an angle of inclination that substantially corresponds with an angle of second outer wall 970 of second plunger 950 relative to a longitudinal axis 901 shown in FIG. 10. Such a corresponding arrangement, depending on a material of which first and/or second plunger 900, 950 are composed (e.g., a soft rubber material), may help avoid a contacting portion of one plunger from compressing and sticking to the other plunger. In other examples, depending on material compositions thereof, such an arrangement may provide a friction fit between first and second plungers 900, 950 may be provided through an engagement between the rim 908 and second outer wall 920. Such a friction fit may enable several plungers to be stacked together and carried without concern for plungers becoming dislodged from one another with applications of relatively weak external forces. On the other hand, this friction fit may not be so strong as to require a large force causing an individual to strain when attempting to separate stacked plungers, such as first and second plungers 900, 950 of FIG. 10.

Turning to a relationship between head 960 of second plunger 950 and first and second recesses 910, 920 defined by first plunger 900. As shown in FIG. 10, a first clearance 990 is provided between an end face 962 of head 960 of second plunger 900, and recess and end surfaces defining second and third recesses 920, 930 of first plunger 900. In addition, a second clearance 995 is defined between plunger protrusions 915 for first plunger 900 and a first outer wall 966 of second plunger 950. Accordingly, in some embodiments, head 960 and plunger protrusions 915 are not and will not be in contact in a stacked arrangement, nor be at risk of making contact. Furthermore, second outer wall 970 does not contact plunger protrusions 915 in the stacked arrangement. Accordingly, in a stacked arrangement, engagement components (e.g., head, second outer wall, plunger protrusions) of example plungers described herein, that if damaged or deformed may impact an ability of an example plunger to operate as expected within an auto injector, are not at risk of coming contact and being changed by such contact.

FIG. 11 is a cross-sectional view of an auto-injector 1000 showing a plunger 1005, according to an aspect of the disclosure. More specifically, FIG. 11 includes a depiction of plunger 1005 disposed in a cartridge 1060 that includes a stopper 1065 positioned between cartridge 1060 and a needle 1004. In some examples, translational movement of cartridge 1060 may be actuated by translational movement of plunger 1005 such that stopper 1065 may be moved into contact with needle 1004. Continued movement of cartridge 1060 (via movement of plunger 1005) may cause needle 1004 to pierce stopper 1065, putting a fluid channel (not shown) within needle 1004 to be in fluid communication with a cavity 1003 defined by an inner wall of cartridge 1060. In some examples, where cavity 1003 includes a fluid (e.g., a medicament), such fluid may flow into the fluid passage of needle 1004 that has pierced stopper 1065, and be injected into an individual on which auto-injector 1000 is positioned.

Translational movement of plunger 1005 may be actuate by a screw assembly 1070 according to an aspect of the present disclosure. Screw assembly 1070 may be substantially similar to exemplary screw assembly 170 of FIG. 1 and include first screw 1080 engaged to plunger, second screw 1090 operatively engaged with first screw 1080, and third screw 1095 operatively engaged with second screw 1090. A drive output 1002 may be operatively coupled to third screw 1095 and a drive mechanism (not shown). In some examples, drive output 1002 includes a first gear that is rotated with an operation of the drive mechanism and corresponding movement (rotational) of a second gear. The second gear being coupled to the drive mechanism and in meshed engagement with first gear of drive output 1002, in one example. In some examples, third screw 1095 may define a key 1096 at a respective distal end, and key 1096 may fit into an engagement slot of drive output 1002. As a result of this engagement, in some examples, rotation of drive output 1002 may cause corresponding rotational movement of third screw 1095.

In some examples, screw assembly 1070 may provide a leadscrew mechanism that is coupled to plunger 1005 and extends axially in a telescoping operation upon relative rotation about a longitudinal axis of cartridge 1060. In some examples, the screw assembly 1070 may have a 100 N output, a 20 mm stroke, and a 7°/45° buttress thread shape with a 0.75 mm pitch. The materials for the first, second, and/or third screws 1085, 1090, 1095 may include acetal and polybutylene terephthalate. As shown, and as discussed above with reference to FIG. 4, screw assembly 1070 may extend within plunger 1005 to reduce dead space behind plunger 1005.

Notably, reference herein to “one example,” or “an example” means that a particular feature, structure, or characteristic described in connection with the example may be included, employed and/or incorporated in one, some or all of the examples of the present disclosure. The usages or appearances of the phrase “in one example” or “in another example” in the specification are not referring to the same example, nor are separate or alternative examples necessarily mutually exclusive of one or more other examples, nor limited to a single exclusive example. The same applies to the terms “implementation,” and “example.” The present disclosure are neither limited to any single aspect nor example thereof, nor to any combinations and/or permutations of such aspects and/or examples. Moreover, each of the aspects of the present disclosure, and/or examples thereof, may be employed alone or in combination with one or more of the other aspects of the present disclosure and/or examples thereof. For the sake of brevity, certain permutations and combinations are not discussed and/or illustrated separately herein.

Further, as indicated above, an aspect or implementation described herein as “exemplary” is not to be construed as preferred or advantageous, for example, over other examples or implementations; rather, it is intended convey or indicate the aspect or aspects are example aspect(s).

The present disclosure is further described by the following non-limiting items:

The present disclosure is further described by the following non-limiting items:

Item 1. A plunger comprising: a body extending from a proximal end to a distal end, the body further including: a head at the proximal end, the head including a first end face and a first outer wall extending from the first end face; and a first recess extending into the body at the distal end, the recess being defined by an inner circumferential wall of the body, wherein the body further includes a plurality of protrusions extending radially inward from the inner circumferential wall.

Item 2. The plunger of item 1, wherein each of the protrusions includes a blunt tip facing the distal end of the body.

Item 3. The plunger of item 2, wherein the blunt tip has a smaller width than a remainder of the protrusion.

Item 4. The plunger of item 2, wherein each protrusion includes a radially-innermost surface, and a first bevel between the radially-innermost surface and the inner circumferential wall of the body.

Item 5. The plunger of item 4, wherein each protrusion includes a second bevel between the first bevel and the inner circumferential wall of the body.

Item 6. The plunger of item 1, wherein circumferentially-adjacent protrusions define a slot between one another.

Item 7. The plunger of item 1, wherein the plunger include 8-10 protrusions.

Item 8. The plunger of item 1, wherein further including a first distally-facing end surface at the proximal end of the first recess.

Item 9. The plunger of item 8, further including a second recess within the body, wherein the second recess is disposed proximally of the first recess.

Item 10. The plunger of item 9, wherein the first recess has a larger diameter than the second recess.

Item 11. The plunger of item 9, wherein the second recess is defined by a second inner circumferential wall of the body.

Item 12. The plunger of item 11, wherein the second inner circumferential wall is free of protrusions.

Item 13. The plunger of item 9, further including a second distally-facing end surface at the proximal end of the second recess.

Item 14. The plunger of item 13, wherein the second distally-facing end surface has a larger width than the first distally-facing end surface.

Item 15. The plunger of item 9, further including a third recess within the body, wherein the third recess is disposed proximally of the second recess.

Item 16. The plunger of item 15, wherein the second recess has a larger diameter than the third recess.

Item 17. The plunger of item 15, wherein the third recess is defined by a third inner circumferential wall of the body.

Item 18. The plunger of item 15, further including a third distally-facing end surface at the proximal end of the third recess.

Item 19. The plunger of item 18, wherein the third distally-facing end surfaces closes the proximal end of the third recess.

Item 20. A medical device comprising: a plunger having a body extending from a proximal end to a distal end, the body further including: a head at the proximal end, the head including a first end face and a first outer wall extending from the first end face; and a first recess extending into the body at the distal end, the recess being defined by an inner circumferential wall of the body, wherein the body further includes a plurality of protrusions extending radially inward from the inner circumferential wall; and a screw configured to extend into the first recess and engage with the inner circumferential wall of the body, wherein the screw includes a plurality of extensions, wherein each extension is disposed between a pair of circumferentially-adjacent protrusions of the plunger.

Item 21. The medical device of item 20, wherein the screw further includes a nose region extending beyond ends of each extension.

Item 22. The medical device of item 20, wherein each of the plurality of extensions includes a tip that is generally triangular in shape.

What is claimed is:

1. A plunger comprising:
a body extending from a proximal end to a distal end, the body further including:
a head at the proximal end, the head including a first end face and a first outer wall extending from the first end face; and
a first recess extending into the body at the distal end, the recess being defined by an inner circumferential wall of the body, wherein the body further includes a plurality of protrusions extending radially inward from the inner circumferential wall.

2. The plunger of claim 1, wherein each of the protrusions includes a blunt tip facing the distal end of the body.

3. The plunger of claim 2, wherein the blunt tip has a smaller width than a remainder of the protrusion.

4. The plunger of claim 2, wherein each protrusion includes a radially-innermost surface, and a first bevel between the radially-innermost surface and the inner circumferential wall of the body.

5. The plunger of claim 4, wherein each protrusion includes a second bevel between the first bevel and the inner circumferential wall of the body.

6. The plunger of claim 1, wherein circumferentially-adjacent protrusions define a slot between one another.

7. The plunger of claim 1, wherein the plunger include 8-10 protrusions.

8. The plunger of claim 1, wherein further including a first distally-facing end surface at the proximal end of the first recess.

9. The plunger of claim 8, further including a second recess within the body, wherein the second recess is disposed proximally of the first recess.

10. The plunger of claim 9, wherein the first recess has a larger diameter than the second recess.

11. The plunger of claim 9, wherein the second recess is defined by a second inner circumferential wall of the body.

12. The plunger of claim 11, wherein the second inner circumferential wall is free of protrusions.

13. The plunger of claim 9, further including a second distally-facing end surface at the proximal end of the second recess.

14. The plunger of claim 13, wherein the second distally-facing end surface has a larger width than the first distally-facing end surface.

15. The plunger of claim 9, further including a third recess within the body, wherein the third recess is disposed proximally of the second recess.

16. The plunger of claim 15, wherein the second recess has a larger diameter than the third recess.

17. The plunger of claim 15, wherein the third recess is defined by a third inner circumferential wall of the body.

18. The plunger of claim 15, further including a third distally-facing end surface at the proximal end of the third recess.

19. The plunger of claim 18, wherein the third distally-facing end surfaces closes the proximal end of the third recess.

20. A medical device comprising:
a plunger having a body extending from a proximal end to a distal end, the body further including:
a head at the proximal end, the head including a first end face and a first outer wall extending from the first end face; and
a first recess extending into the body at the distal end, the recess being defined by an inner circumferential wall of the body, wherein the body further includes a plurality of protrusions extending radially inward from the inner circumferential wall; and
a screw configured to extend into the first recess and engage with the inner circumferential wall of the body, wherein the screw includes a plurality of extensions, wherein each extension is disposed between a pair of circumferentially-adjacent protrusions of the plunger.

21. The medical device of claim 20, wherein the screw further includes a nose region extending beyond ends of each extension.

22. The medical device of claim 20, wherein each of the plurality of extensions includes a tip that is generally triangular in shape.
